(19) United States
(12) Patent Application Publication
(10) Pub. No.: US 2023/0190195 A1
(43) Pub. Date: Jun. 22, 2023

(54) INTRAORAL MONITORING DEVICE, SYSTEM, AND METHOD

(71) Applicant: Dianyx Innovations, LLC, Westerville, OH (US)

(72) Inventors: Robert Kibler, Columbus, OH (US); Seyed Nabavi, Montreal (CA); John Cogan, Blackrock County (IE); Asim Roy, Dublin, OH (US); Brandon Canfield, Columbus, OH (US); Collin Emerick, columbus, OH (US)

(73) Assignee: Dianyx Innovations, LLC, Westerville, OH (US)

(21) Appl. No.: 18/069,302

(22) Filed: Dec. 21, 2022

Related U.S. Application Data
(60) Provisional application No. 63/292,426, filed on Dec. 22, 2021.

(51) Int. Cl. A61B 5/00; A61B 5/0205

(52) U.S. Cl. CPC A61B 5/682; A61B 5/02055; A61B 5/002; A61B 5/4812; A61B 2562/164; A61B 2562/166; A61B 5/02433

(57) ABSTRACT

An intraoral monitoring device includes a plurality of sensors integrated on a flexible substrate. The plurality of sensors includes first and second photoplethysmography (PPG) sensors. The first PPG sensor is coupled along an anterior portion of the flexible substrate and adapted to be located intraorally along a first anatomical location defined along a mid-line of a maxilla, facing the labial mucosa, directly at a septal branch of a superior labial artery in a front lip to consistently provide a plurality of first PPG signal, relating to a plurality of cardiorespiratory parameters. Further, the second PPG sensor is coupled to a rear portion of the flexible substrate, adapted to be located intraorally along a second anatomical location defined along the interior part of an oral cavity facing outward toward the labial mucosa to consistently provide a plurality of second PPG signals, relating to a plurality of muscular parameters.

### CROSS-REFERENCE TO RELATED APPLICATIONS

This application claims the benefit of U.S. Provisional Application No. 63/292,426 filed Dec. 22, 2022, which is incorporated herein by reference as if fully set forth herein.

### FIELD OF INVENTION

The present invention relates to the medical device industry, and, more particularly, to an intraoral monitoring device for monitoring, measuring, and reporting sleep-related data of a user.

### BACKGROUND OF THE INVENTION

Sleep is an essential requirement for human survival. However, for many people diagnosed with a sleep disorder, going to sleep can be a dreaded experience due to the lack of restful sleep. For example, one of the sleep disorders may include sleep apnea, which is a relatively common and potentially life-threatening sleep disorder during which a person experiences one or more pauses in breathing, and in some instances, experiences shallow breaths during sleep. Fortunately, the brain usually detects such an inability to breathe and briefly awakens the individual to reopen the airway. Unfortunately, these continuous disruptions in breathing have also been associated with increased blood pressure, stroke, and diabetes as well as other chronic disorders including death.

Further, there are also related intraoral muscular disorders, such as abnormal jaw movement which are associated with mouth, teeth, and jaw pathologies. Jaw movement abnormalities such as bruxism (teeth grinding) may lead to tooth decline or muscular tension, which may lead to a stiff jaw or headaches and even to changes in the structural features of the face.

These disorders may be currently treated by methods such as surgery or by any oral appliance or implantable devices, or a combination involving several other methods. Due to the variety of abnormalities that cause these disorders and individual needs and preferences, however, no single solution has been found to be acceptable to all who suffer from one or more such sleep disorders.

Accordingly, there exists a need for proper monitoring of intraoral activities to take appropriate steps in advance for the detection of any disorders, including, but not limited to, sleep disorders or intraoral muscular disorders, or for that matter monitoring of overall intraoral activities on a daily and real-time basis.

### SUMMARY OF THE INVENTION

An intraoral monitoring device, system, and method for obtaining a sleep pattern of a user overcomes the above drawbacks and may provide an advance for the detection of any disorders, including, but not limited to, sleep disorders or intraoral muscular disorders, or for that matter, monitoring of overall intraoral activities on a daily and real-time basis.

The intraoral monitoring device may be coupled with a mouthpiece that is adapted to be received intraorally on the dentition of a user. The intraoral monitoring device may itself be a mouthpiece, which may be received intraorally in a dentition of a user. The intraoral monitoring device may include a flexible substrate, and a plurality of sensors integrated on a flexible printed circuit board (PCB) and coupled to the flexible substrate.

The plurality of sensors may include a first photoplethysmography (PPG) sensor and a second PPG sensor. The first PPG sensor may be coupled along the anterior portion of the flexible substrate and adapted to be located intraorally along a first anatomical location defined along a mid-line of a maxilla, facing outward to a labial mucosa, directly at a septal branch of a superior labial artery in a front lip. Such a location of the first PPG sensor, intraorally, may be capable of consistently providing a plurality of first PPG signal, during sleep of the user, relating to a plurality of cardiorespiratory parameters including a heart rate, a respiratory rate, and a blood oxygen saturation of the user. Further, the second PPG sensor may be coupled to the posterior portion of the flexible substrate, adapted to be located intraorally along a second anatomical location defined along the interior part of an oral cavity facing outward toward the labial mucosa. This second PPG sensor location may be capable of consistently providing a plurality of second PPG signals, during the user's sleep, relating to a plurality of muscular parameters, including from the Buccinator, the Medial Pterygoid, and the Masseter muscles. The plurality of muscular parameters based on the plurality of second PPG signals may be configured to classify one or more sleep stages of the user in combination with the plurality of cardiorespiratory parameters based on the plurality of first PPG signal, as the plurality of muscular parameters varies according to the one or more sleep stages, as well as other information related to muscle activity.

The first PPG sensor may include at least one first light or infrared (IR) source, and at least one first photodetector. The at least one first light or IR source is configured to emit light along the first anatomical location. Further, the at least one first photodetector is operatively coupled to the at least one first light or infrared (IR) source to measure the light reflected from the first anatomical location to generate the plurality of first PPG signal based on the wavelength of the light reflected to determine the plurality of cardiorespiratory parameters.

Similarly, the second PPG sensor may include at least one second light or IR source, and at least one second photodetector. The at least one second light or IR source may be configured to emit light along the second anatomical location. Further, the at least one second photodetector operatively coupled to the at least one second light or IR source to measure the light reflected from the second anatomical location to generate the plurality of second PPG signal based on the wavelength of the light reflected to determine the plurality of muscular parameters.

The plurality of sensors may further include at least one microphone or acoustic sensor coupled along a first side portion of the flexible substrate and adapted to be located intraorally along a third anatomical location defined along the interior part of the oral cavity near the soft palate and the uvula, where an airway collapses and sound creates. The at least one microphone or acoustic sensor is capable of measuring the snoring intensity of the sleeping user.

The plurality of sensors may further include at least one pH sensor coupled on the flexible substrate proximate to the at least one microphone or acoustic sensor and adapted to be located intraorally along a fourth anatomical location, proximate to the third anatomical location defined within the oral cavity of the user along the maxilla closest to the salivary glands/ducts, whereby saliva exists in a substantially higher volume. The at least one pH sensor is capable of measuring the pH level of the saliva to monitor overall sleep quality during sleep.

The plurality of sensors may further include at least one temperature sensor; at least one pressure sensor; and at least one accelerometer. Each of the at least one temperature sensor, the at least one pressure sensor, and the at least one accelerometer may be coupled along a second side portion of the flexible substrate.

The intraoral monitoring device may further include a microcontroller integrated with the flexible printed circuit board (PCB) on the flexible substrate to be communicably coupled to the plurality of sensors to analyze data received by the plurality of sensors.

The intraoral monitoring device may also include a Bluetooth module integrated into the flexible printed circuit board (PCB) and coupled to the flexible substrate, proximate to the first PPG sensor, and adapted to be located intraorally along a fifth anatomical location defined along the center of an oral cavity where an amount of saliva is substantially low to transmit the data received by the plurality of sensors or the microcontroller to an external computing device.

The intraoral monitoring device may also include a battery, and a charging circuitry electrically coupled to the battery. The battery and the charging circuitry may be coupled to the flexible substrate and integrated with the PCB and adapted to be located intraorally along a sixth anatomical location defined along a distal portion of the maxillary dentition.

A method for obtaining a sleep pattern of a user may include: inserting, intraorally, a mouthpiece having an intraoral monitoring device, wherein the intraoral monitoring device comprises a flexible substrate, and a plurality of sensors integrated on a flexible printed circuit board (PCB) and coupled to the flexible substrate, the plurality of sensors comprising: a first photoplethysmography (PPG) sensor coupled along an anterior portion of the flexible substrate, and a second photoplethysmography (PPG) sensor coupled to a posterior, portion of the flexible substrate; placing, intraorally, the first PPG sensor along a first anatomical location defined along a mid-line of a maxilla, facing the labial mucosa, directly at a septal branch of a superior labial artery in a front lip; obtaining a plurality of first PPG signal from the first PPG sensor, consistently, during sleep of the user relating to a plurality of cardiorespiratory parameters including heart rate, respiratory rate, and blood oxygen saturation of the user; placing, intraorally, the second PPG sensor along a second anatomical location defined along the interior part of an oral cavity facing outward toward the labial mucosa; obtaining a plurality of second PPG signals from the second PPG sensor, consistently, during sleep of the user relating to a plurality of muscular parameters, including from the Buccinator, the Medial Pterygoid, and the Masseter muscles of the user; and classifying one or more sleep stages of the user based on the plurality of muscular parameters based on the plurality of second PPG signals in combination with the plurality of cardiorespiratory parameters based on the plurality of first PPG signal, as the plurality of muscular parameters varies according to the one or more sleep stages.

A system for obtaining a sleep pattern of a user may include a network, one or more computing devices or platforms communicably coupled to the network, and an intraoral monitoring device as summarized above.

### BRIEF DESCRIPTION OF THE DRAWINGS

FIG. 1 illustrates a schematic of an intraoral monitoring device depicting a plurality of sensors arrangement with the demonstration of its placement in the oral cavity.

FIG. 2 illustrates a backside view of an intraoral monitoring device depicting a plurality of sensors arrangement with the demonstration of its ability to bend.

FIGS. 3A and 3B, respectively, illustrate block diagrams of a first PPG sensor and a second PPG sensor.

FIG. 4 depicts a working mechanism flowchart of an intraoral monitoring device with a plurality of sensors arrangement.

FIG. 5 illustrates a graph depicting intraorally measured PPG signals at red and IR wavelengths.

FIG. 6 illustrates a graph depicting estimated HR, SpO2, and RR from the intraorally measured PPG signals using a first photoplethysmography (PPG) sensor.

FIG. 7 illustrates a graph depicting intraorally monitoring masseter muscle activity by using a second photoplethysmography (PPG) sensor.

FIG. 8 illustrates an intraoral monitoring device depicting a plurality of sensors arrangement attached to an oral appliance.

FIG. 9 illustrates a graph depicting muscle activity measured by the oral appliance demonstrated in FIG. 8.

FIG. 10 depicts a schematic system diagram outlining software architecture to analyse, transmit, and share data captured by an intraoral monitoring device depicting a plurality of sensors arrangement.

### DETAILED DESCRIPTION OF THE INVENTION

The following is a detailed description of embodiments of the invention depicted in the accompanying drawings.

The terms “a” and “an” herein do not denote a limitation of quantity, but rather denote the presence of at least one of the referenced items. Thus, for example, a reference to “a prosthesis” includes one or more prostheses.

The terms “having”, “comprising”, “including”, and variations thereof signify the presence of a component.

An intraoral monitoring device may be coupled with a mouthpiece that is adapted to be received intraorally in a dentition of a user. The intraoral monitoring device may itself be a mouthpiece, which may be received intraorally on the dentition of a user. in a dentition of a user. The intraoral monitoring device may simply act as a monitoring device that may be used with non-custom oral appliances, essentially, for an over-the-counter solution. The intraoral monitoring device includes a flexible substrate, and a plurality of sensors integrated on a flexible printed circuit board (PCB) and coupled to the flexible substrate. The plurality of sensors may include a first PPG sensor, and a second PPG sensor. The first PPG sensor may be coupled along an anterior portion of the flexible substrate and adapted to be located intraorally along a first anatomical location defined along a mid-line of a maxilla, facing the labial mucosa, directly at a septal branch of a superior labial artery in a front lip. Such a location of the first PPG sensor, intraorally, may be capable of consistently providing a plurality of first PPG signal, during sleep of the user, relating to a plurality of cardiorespiratory parameters including a heart rate, a respiratory rate, and a blood oxygen saturation of the user.

Further, the second PPG sensor may be coupled to a rear portion of the flexible substrate, adapted to be located intraorally along a second anatomical location defined along the interior part of an oral cavity facing outward toward the labial mucosa. Such a location of the second PPG sensor, intraorally, may be capable of consistently providing a plurality of second PPG signals, during user's sleep, relating to a plurality of muscular parameters, including from the Buccinator, the Medial Pterygoid, and the Masseter muscles of the user. The plurality of muscular parameters based on the plurality of second PPG signals is configured to classify one or more sleep stages of the user in combination with the plurality of cardiorespiratory parameters based on the plurality of first PPG signal, as the plurality of muscular parameters varies according to the one or more sleep stages.

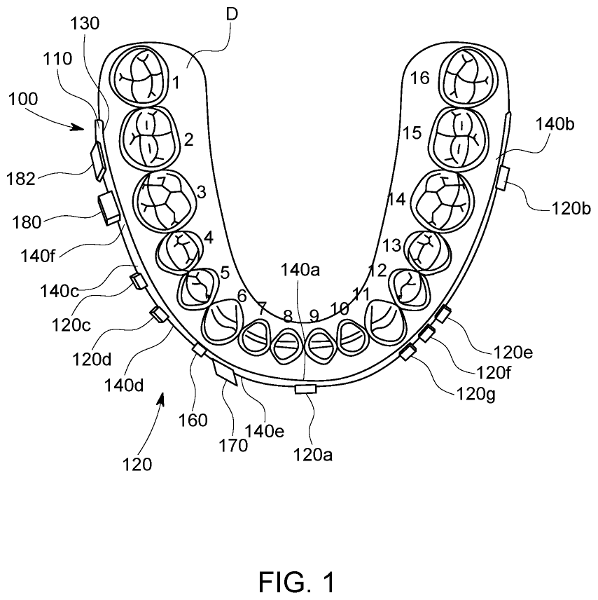

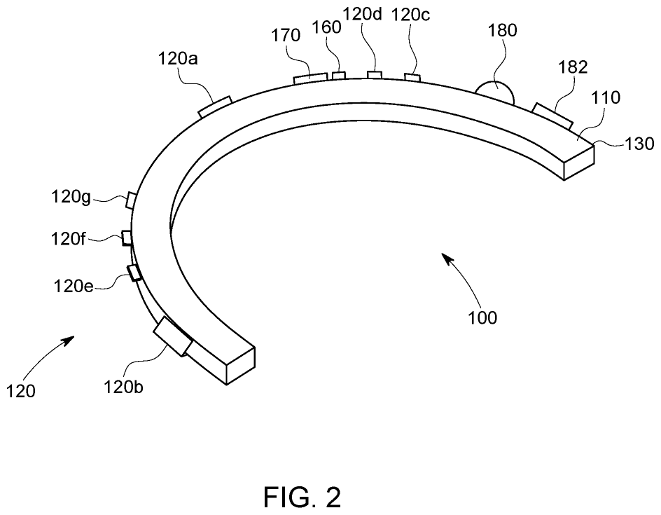

FIGS. 1 and 2 will now be referred to describe an intraoral monitoring device 100. FIG. 1 illustrates a schematic of the intraoral monitoring device 100 depicting a plurality of sensors 120 arrangement with the demonstration of its placement in a dentition (D) of a user in the oral cavity, and FIG. 2 illustrates a backside view of the intraoral monitoring device 100 depicting a plurality of sensors 120 arrangement with the demonstration of its ability to bend.

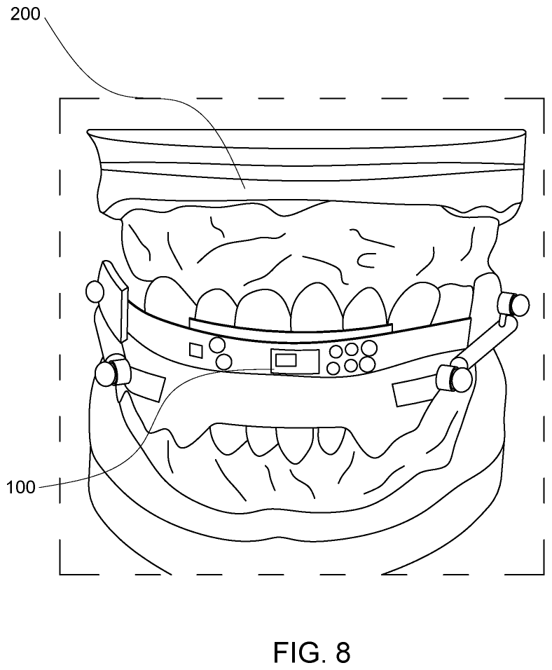

The intraoral monitoring device 100 may be placed in a dentition (D) of a user in the oral cavity. In another embodiment, as seen in FIG. 8, the intraoral monitoring device 100 may be coupled with a mouthpiece 200. The intraoral monitoring device 100 along with the mouthpiece 200 may be adapted to be received intraorally in a dentition of a user. The intraoral monitoring device 100 may include a flexible substrate 110 having a flexible enough to accommodate itself to various designs of a mouthpiece, such as, the mouthpiece 200. The flexible substrate 110 may be made of flexible material, including, but not limited to, nylon, or other thin thermoplastics. The flexible substrate 110 may also be made of silicon material. The flexible material can be selected from various other material as commonly used in the dental field. The intraoral monitoring device 100 may further include a plurality of sensors 120 integrated on a flexible printed circuit board (PCB) 130 and coupled to the flexible substrate 110. The plurality of sensors (may also be referred to as a sensor bundle) includes a set of different sensors, each of which can have digital or analogue output signals.

The plurality of sensors 120 may include a first photoplethysmography (PPG) sensor 120a, and a second photoplethysmography (PPG) sensor 120b. The first PPG sensor 120a may be coupled along an anterior portion of the flexible substrate 110, and adapted to be located intraorally along a first anatomical location 140a. The first anatomical location 140a may be defined along a mid-line of a maxilla, facing outward the labial mucosa, directly at a septal branch of a superior labial artery in a front lip. Such first anatomical location 140a may be seen along teeth no. 8 and 9. Such first anatomical location 140a of the first PPG sensor 140a, intraorally, may be capable of consistently providing a plurality of first PPG signal, during sleep of the user, relating to a plurality of cardiorespiratory parameters including a heart rate (HR), a respiratory rate (RR), and a blood oxygen saturation (SpO₂) of the user.

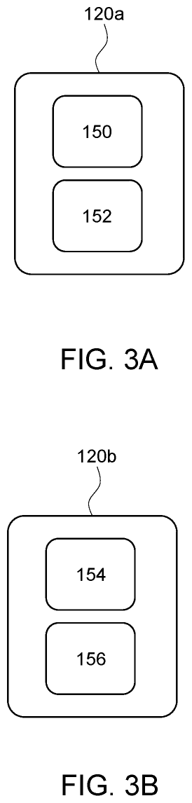

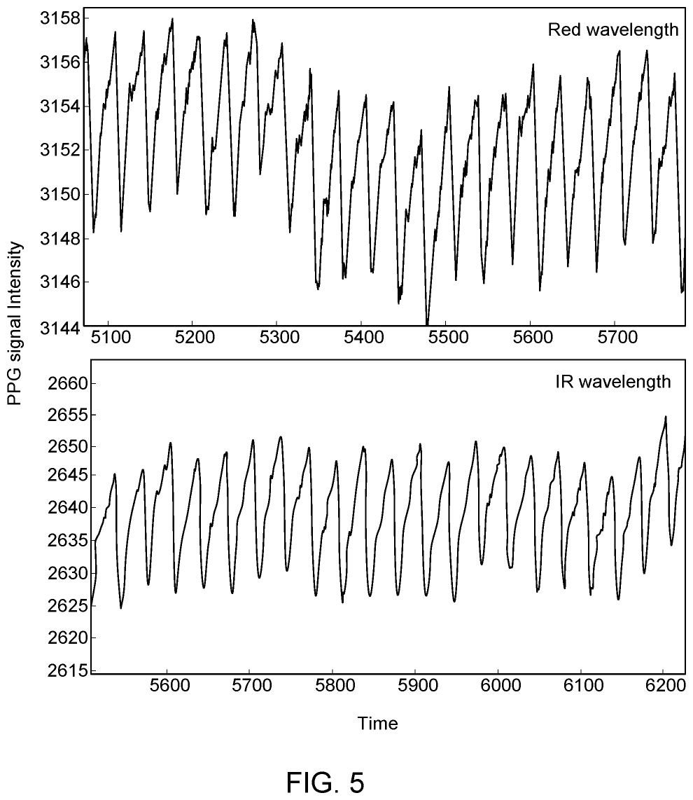

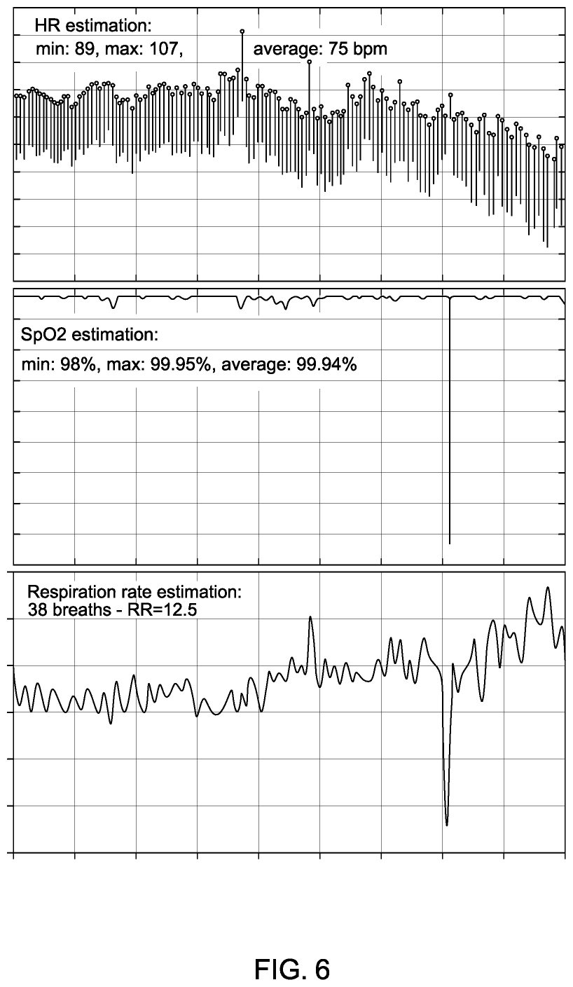

As seen in FIG. 3A the first PPG sensor 120a may include at least one first light or infrared (IR) source 150, and at least one first photodetector 152. The first light or infrared (IR) source 150 may be configured to emit light along the first anatomical location 140a. The first light or infrared (IR) source 150 may include two or three source lights (LEDs) at different wavelengths, i.e., red, infrared (IR), or green. Further, the first photodetector 152 is operatively coupled to the first light or IR source 150. Thus, the reflection/transmission of the source light from/through the skin at the first anatomical location 140a is measured by the photodetector 152 to generate the plurality of first PPG signal based on the wavelength of the light reflected to determine the plurality of cardiorespiratory parameters. Because PPG-based detection is a relatively inexpensive technique and does not require an additional measurement procedure, it is widely used to provide portable sensor platforms for various healthcare monitoring. It has been shown that the PPG signals after processing provides substantial information regarding HR, RR, and SpO₂. It is worth pointing out that a topology of PPG signals, including but not limited to, amplitude, period, frequency, and variation, can be used to identify the different stages of sleep. Furthermore, the PPG data (single channel or dual channel) measured from the first anatomical location 140a after deploying some signal processing techniques (denoising, averaging, etc.) can be used to determine different stages of sleep. The cardiorespiratory parameters of HR, RR, and SpO₂ obtained by processing the PPG signals shown in FIG. 5 are illustrated in FIG. 6.

Further, the second PPG sensor 120b may be coupled to a rear portion of the flexible substrate 110, adapted to be located intraorally along a second anatomical location 140b. The second anatomical location 140b may be defined along the interior part of an oral cavity facing outward toward the labial mucosa. Such second anatomical location 140b may be seen in FIG. 1 along teeth no. 1-3 or along teeth no. 14-16. Such second anatomical location 140b of the second PPG sensor 120b, intraorally, may be capable of consistently providing a plurality of second PPG signals, during the sleep of the user, relating to a plurality of muscular parameters, including from the Buccinator, the Medial Pterygoid, and the Masseter muscles of the user.

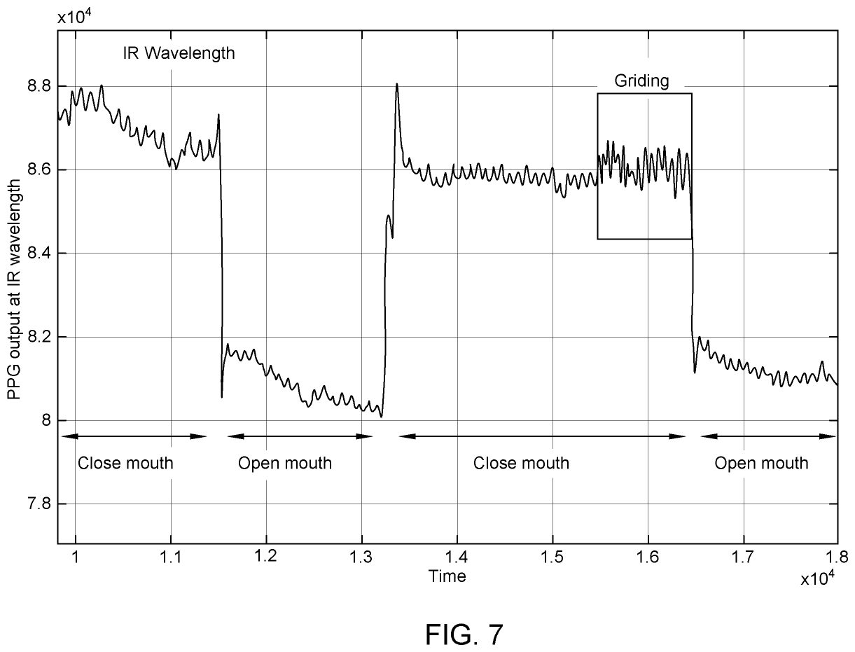

As seen in FIG. 3B, the second PPG sensor 120b may include at least one second light or infrared (IR) source 154, and at least one second photodetector 156. The second light or infrared (IR) source may be configured to emit light along the second anatomical location 140b. The second light or infrared (IR) source 150 may include two or three source lights (LEDs) at different wavelengths, i.e., red, infrared (IR), or green. Further, the second photodetector 156 may be operatively coupled to the second light or infrared (IR) source. Thus, the reflection/transmission of the source light from/through the skin at the second anatomical location 140b is measured by the photodetector 156 to generate the plurality of second PPG signal based on the wavelength of the light reflected to determine the plurality of muscular parameters. Furthermore, the PPG data (single channel or dual channel) measured from the second anatomical location 140a after deploying some signal processing techniques (denoising, averaging, etc.) can be used to determine different muscular activities during various stages of sleep. The muscular parameters from the Buccinator, the Medial Pterygoid, and the Masseter muscles obtained by processing the second PPG signals, as shown in FIG. 7.

The plurality of muscular parameters based on the plurality of second PPG signals is configured to classify one or more sleep stages of the user in combination with the plurality of cardiorespiratory parameters based on the plurality of first PPG signal, as the plurality of muscular parameters varies according to the one or more sleep stages.

As further seen in FIGS. 1 and 2, in one arrangement, the plurality of sensors 120 may further include at least one microphone or acoustic sensor 120c coupled along a first side portion of the flexible substrate 110, and adapted to be located intraorally along a third anatomical location 140c. In one embodiment, the third anatomical location 140c may be defined along the interior part of the oral cavity near the soft palate and the uvula, where an airway collapses and sound creates. Such third anatomical location 140c may be seen in FIG. 1 along teeth no. 4 or along teeth no. 13. The microphone or acoustic sensor 120c may be a low-power microphone capable of measuring the snoring intensity of the user, while the user is sleeping. Snoring is the harsh sound that occurs when air passes through the relaxed tissues of the throat. Snoring is usually associated with sleep disorders. Therefore, the audio signal captured by the microphone or acoustic sensor 120c may also be used to determine the sleep quality.

The plurality of sensors 120 may further include at least one pH sensor 120d coupled on the flexible substrate 110 proximate to the microphone or acoustic sensor 120c and adapted to be located intraorally along a fourth anatomical location 140d, proximate to the third anatomical location 140c. The fourth anatomical location 140d may be defined within the oral cavity of the user along the maxilla arch closest to the salivary glands/ducts, whereby saliva exists in a substantially higher volume. Such fourth anatomical location 140d may be seen in FIG. 1 along teeth no. 5. The pH sensor 120d may be capable of measuring the pH level of the saliva to monitor overall sleep quality, while the user is sleeping. It is widely discussed that intraoral pH has circadian rhythms, and its level varies significantly during sleep and wakefulness. As such, the intraorally measured pH level can be considered as another parameter to monitor sleep quality.

The plurality of sensors 120 may further include at least one temperature sensor 120e, at least one pressure sensor 120f, and at least one accelerometer 120g. In one example arrangement, as seen in FIGS. 1 and 2, the temperature sensor 120e, the pressure sensor 120f, and the accelerometer 120g may be coupled along a second side portion of the flexible substrate, for example, as shown to be disposed between the first PPG sensor 120a and the second PPG sensor 120b, in FIG. 1. However, without departing from the scope of the present disclosure, the position of the temperature sensor 120e, the pressure sensor 120f, and the accelerometer 120g on the flexible substrate 110 may not be limited to a specific location, and may vary as per the convenient of the manufacturing process or can be placed at any available empty spot on the flexible substrate. The temperature sensor 120e measures the body temperature that varies during each stage of sleep. On the other hand, the respiratory route (i.e., nasal or oral) has an impact on the local temperature of the oral cavity.

In this regard, the temperature sensor 120e may measure the temperature of the surrounding area of the oral cavity as well as the core body. Such measured body temperature levels combined with other measured parameters may be used to assess sleep quality. Furthermore, the pressure sensor 120f measures the intraoral pressure by the measurement of airflow during inhalation and exhalation. As is known, pressure and volume are inversely proportional, therefore, the volume of air inside the oral cavity, which is a function of the airflow from the nasal passage, can be easily estimated based on the output of the pressure sensor. Moreover, the accelerometer 120g may be a three-axis accelerometer for actigraphy. Actigraphy monitors movement and may be used to assess sleep parameters, such as sleep-wake cycles. The output of the accelerometer 120g may determine the user's head and body position, namely supine, prone, right side, and left side. In addition, the intraorally measured accelerometer output signal may be used for sensor fusion techniques, which remove motion artifacts from the first and second PPG signals received from the first PPG sensor 120a and the second PPG2 sensor 120b.

The intraoral monitoring device 100 may further include a microcontroller 160 integrated with the flexible PCB 130 on the flexible substrate 110 to be communicably coupled to the plurality of sensors, such as the first PPG sensor 120a, the second PPG2 sensor 120b, the microphone or acoustic sensor 120c, the pH sensor 120d, the temperature sensor 120e, the pressure sensor 120f, and the accelerometer 120g to analyze data received by such sensors. The intraoral monitoring device 100 may also include a Bluetooth module 170 integrated on the flexible PCB 130 and coupled to the flexible substrate 110, proximate to the first PPG sensor 120a, and adapted to be located intraorally along a fifth anatomical location 140e. The fifth anatomical location 140e may be defined along the center of an oral cavity where the amount of saliva is substantially low (actually, saliva is a salty liquid that is known to be a lossy medium for the propagation of high-frequency waves (e.g., Radio Frequency (RF)) to transmit the data received by the plurality of sensors 120 or the microcontroller to 160 an external computing device. Such fifth anatomical location 140e may be seen in FIG. 1 along teeth no. 6 or 7. Such a placement does not affect the strength of the Bluetooth signal produced by the Bluetooth module 170. In addition, the skin at this location is thinner and does not block the transmission of the Bluetooth signals. The placement of the Bluetooth module at this location helps to ensure that minimal power is consumed for data transmission, which ultimately leads to an increase in the operating time of the intraoral monitoring device 100.

The intraoral monitoring device 100 may also include a battery (or a rechargeable battery) 180, and a charging circuitry 182 electrically coupled to the battery 180. The battery 180 and the charging circuitry 182 may be coupled to the flexible substrate 110 and integrated with the PCB 130, and adapted to be located intraorally along a sixth anatomical location 140f. The sixth anatomical location 140f may be defined along a distal portion of the maxillary dentition to the intraoral monitoring device 100. Due to the large physical aspect of battery 180 and its charging circuitry 182 when compared to other components, placement at the end of the oral cavity (along the sixth anatomical location 140f) is appropriate due to the special anatomy of the mouth, where there is a large area at this point and its skin can be stretched without pain. On the other hand, because of the nature of the battery and its associated circuitry, external pressure created by the sleeping position of wearers has no impact on the overall performance of the monitoring system.

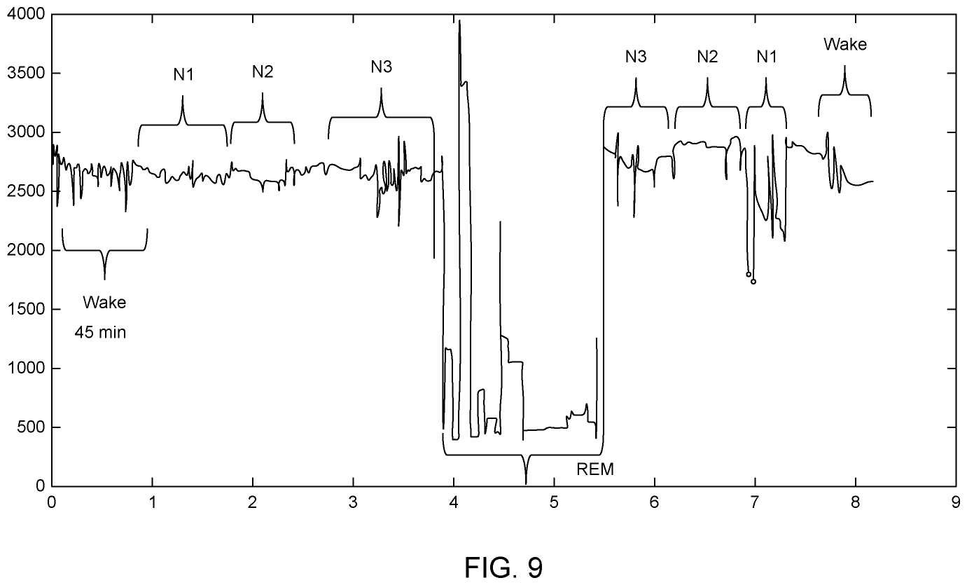

The intraoral monitoring device 100 may be placed in the dentition (D) of a user in the oral cavity as mentioned above. As seen in FIG. 8, the intraoral monitoring device 100 may be coupled with the mouthpiece 200 and then placed in the dentition (D) of a user in the oral cavity. FIG. 9 illustrates a graph depicting muscle activity measured by the mouthpiece 200 or any oral appliance demonstrated in FIG. 8.

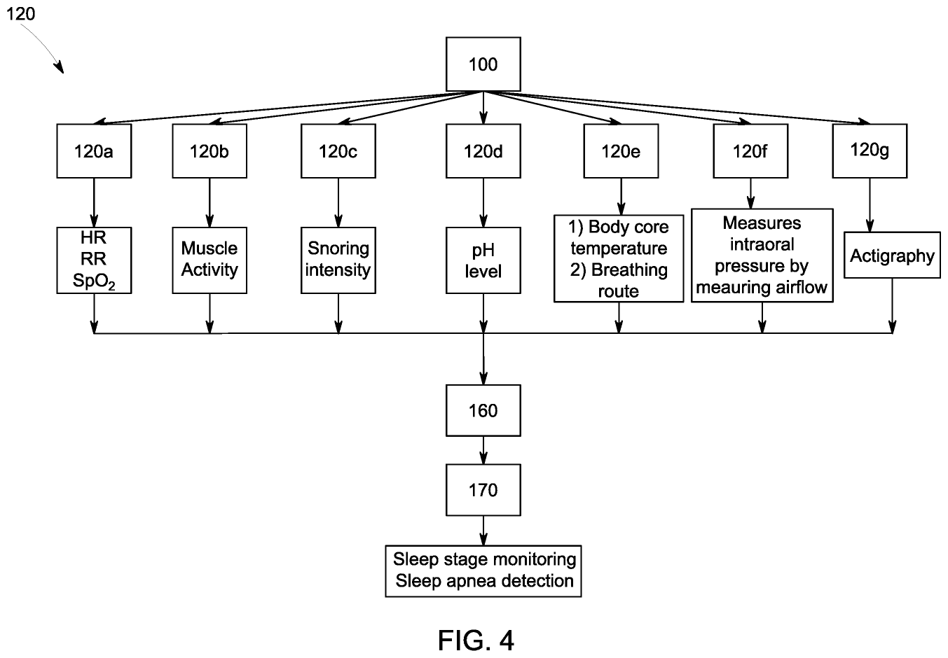

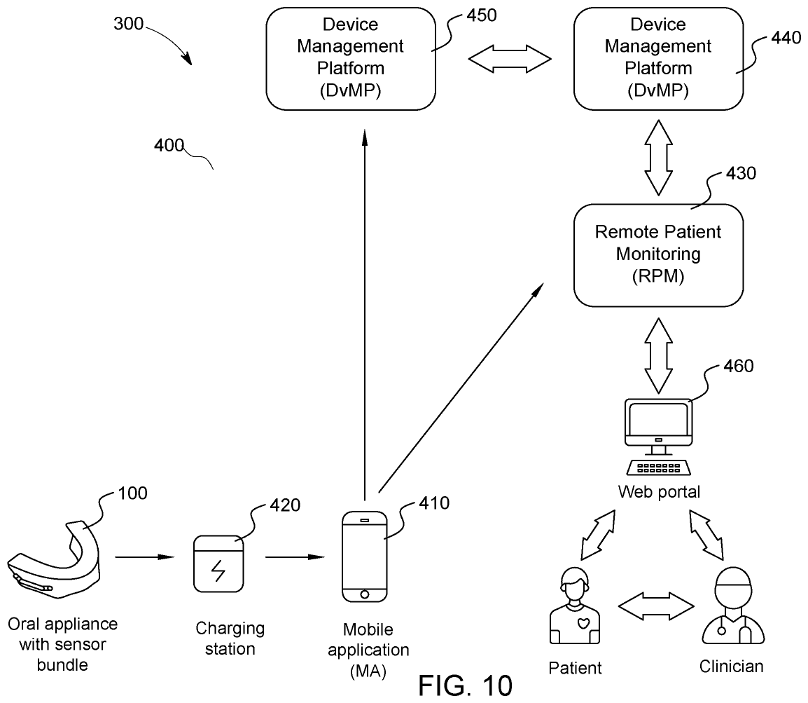

The working principle of the intraoral monitoring device 100 is depicted in FIG. 4. Once the intraoral monitoring device 100 is placed in the dentition (D), independently or via the mouthpiece 200, such that the sensors are aligned along the above-described locations, respective parameters in the form of the output signals from each of the sensors 120, such as the first PPG sensor 120a, the second PPG2 sensor 120b, the microphone or acoustic sensor 120c, the pH sensor 120d, the temperature sensor 120e, the pressure sensor 120f, and the accelerometer 120g may be read by the microcontroller 160 and then sent to the external computing device, such as a personal computer, or a smart cell phone via the Bluetooth module 170 using a network as depicted in FIG. 10. If the connection between the intraoral monitoring device 100 and station is interrupted, the measured data may be temporarily stored in the microcontroller's memory or on-board flash memory. Once a stable connection is re-established between the intraoral monitoring device 100 and the receiving station, real-time data transmission may be resumed.

FIG. 10 shows a schematic system diagram of a system 300 outlining software architecture to analyze, transmit, and share data captured by the intraoral monitoring device 100 with the plurality of sensors arrangement 120. The system 300 includes a network 400 having an architecture, which may include a Mobile App (MA) 410, a Charging Station 420, a Remote Patient Monitoring Platform (RPM) 430, a Data Management Platform (DMP) 440, and a Device Management Platform (DVMP) 450, a Web platform (RPM) 460. The intraoral monitoring device 100 may be coupled to the network 400.

Data captured by the intraoral monitoring device 100 may include, but not be limited to, the following: Heart rate (HR), respiratory rate (RR), oxygen saturation (SpO2), muscle activity, actigraphy, airflow, body temperature, breathing route, pH level, and snoring intensity. These data may be collected by the various sensors 120, as described above and outlined in FIGS. 1 and 2, and processed in accordance with the flowchart as seen in FIG. 4. Once the data is collected, it will be processed either within the intraoral monitoring device 100 itself, or such data may be passed through the MA 410 to the RPM 430, 460, or the DMP 440, DVMP 450.

In the above scenario, certain portions of data may be processed within the intraoral monitoring device 100 itself as purpose, intent, and systematic efficiency dictate. In the above scenario, certain portions of data may be processed the RPM 430, 460 or to the DMP 440, DVMP 450, respective to systematic efficiency and permissions; whereas certain portions of data may or may not be immediately or otherwise available to users accessing the RPM 430, 460, or to the DMP 440, DVMP 450. Once the data is processed, the processed data may be available to approved parties and for authorized intent.

The mobile application (MA) 410 may include a downloadable interface to send and receive user data throughout the system. An example could be, receiving data from the intraoral monitoring device 100 and transmitting it to the DVMP 450 and RPM 430, or receiving data from the DVMP 450 or RPM 430, 460. Users may view and share data appropriately, including outside the present system in any number of categories, including but not limited to permissive users, other users of the present system, applications, platforms, clinicians, organizations, and others. Unique users give the present system initial consent to collect data via this interface. Only after this consent will the present system begin collecting data. The MA 410 is designed with the protocols for maintaining the life of the data within the present system.

The Charging Station 420 may be a storage apparatus used in recharging the battery 180 of the intraoral monitoring device 100 and transmitting collected data. Portions of the data may be transmitted during sleep or charging and in accordance with the protocols.

The DVMP 450 may be a cloud-based architecture that adheres to the protocols. The DVMP 450 may act as a broker, sharing appropriate portions of data with other pieces of the present system. For example, the DVMP 450 receives indicated data from the MA 410 and relays it to the DMP 440. The DVMP 450 also receives indicated data from the DMP 440 and relays it to the MA 410 appropriately and securely. Some portion of productive analysis may occur here.

The DMP 440 may include a cloud-based architecture consisting of computational products with the following capabilities, including but not limited to productive analysis via algorithm and other proprietary methods, report generating, data sharing, machine learning attributes, short-term storage, long-term storage, and integration with authorized applications, platforms, and application program interfaces (API) as well service providers such as electronic health records (EHR) systems, and blockchain systems.

The RPM 430, 460 may include a cloud-based architecture consisting of computational products with the following capabilities, including, but not limited to productive analysis via algorithm and other proprietary methods, report generating, data sharing, short-term data storage, and long-term data storage. The RPM 430, 460 may also include a cloud-based web application accessible by permissive users, clinicians, organizations, and third parties. Permissive users will be able to view, evaluate, manage, and analyze user data for the chief purpose of disease management, therapeutic adherence, and efficacy, via reporting and testing measures.

In the present system, there may be one or more kinds of users, for example, a Unique User, a Permissive User, Clinician(s), and Third Party(ies). The Unique User may be a consenting individual assigned (and confirmed) to the intraoral monitoring device 100. The User may share, assign access, and allow other users, permissive Users, Clinicians, Organizations, Third Party(ies) to access, evaluate, share, view, and distribute the information collected, processed, and analysed by the present technology.

The Permissive User may be a consenting individual, application, program, organization, or platform assigned, permitted by the User to access the Data collected by the intraoral monitoring device 100. Permissive Users shall not unlawfully or unethically collect, share, distribute, disseminate, or sell User Data.

The Clinician(s) may be the consenting party, or parties, indicated for access to User Data to view, evaluate, and manage the accessed information for purposes including but not limited to ensuring usage and compliance, therapeutic efficaciousness, disease management, and improvement, monitoring behavior(s) related to sleep and wake, productive analysis, and any purpose related to health and wellness of the User, whether interacting in-person or remotely via approved application or platform, securely.

The Third Party(ies) may be consenting applications, individuals, organizations, platforms (or other) indicated for access to User Data to view, evaluate, and manage the accessed information for purposes including but not limited to ensuring usage and compliance, therapeutic efficaciousness, disease management, and improvement, monitoring behavior(s) related to sleep and wake, productive analysis, and any purpose related to health and wellness of the User, whether interacting in-person or remotely via approved application or platform, securely

Further, the security of each User and their relative data is sensitive and thus, the present system, may at all phases meet the requirements set forth by the Health Insurance Portability and Accountability Act (HIPPA) of 1996. The requirements herein are met at varying levels by employing the following steps and strategies: It should be noted that information collected by the intraoral monitoring device 100 may be done with consent from the unique user. When intraoral monitoring device 100 is manufactured independently it is assigned an identifier, serial number. The intraoral monitoring device 100 that is paired with an intraoral device creates an exclusive, identifiable relationship between the intraoral monitoring device 100 and the unique user of the intraoral device. Each unique user is required to give consent for the present system to collect, transmit, and analyze. Users with authorized permission(s) to collect, view, evaluate, share, distribute, and analyze unique User Data may be allowed. Data transmission from any portion of the present system to another may be encrypted throughout the entirety of its life within the present system.

The foregoing descriptions of specific embodiments of the present disclosure have been presented for purposes of illustration and description. They are not intended to be exhaustive or to limit the present disclosure to the precise forms disclosed, and many modifications and variations are possible in light of the above teaching. The embodiments were chosen and described to best explain the principles of the present disclosure and its practical application, and to thereby enable others skilled in the art to best use the present disclosure and various embodiments with various modifications as are suited to the particular use contemplated. It is understood that various omissions and substitutions of equivalents are contemplated as circumstances may suggest or render expedient, but such omissions and substitutions are intended to cover the application or implementation without departing from the spirit or scope of the present disclosure.

What is claimed is:

1. An intraoral monitoring device for a mouthpiece adapted to be received intraorally in a dentition of a user, the intraoral monitoring device comprising:
a flexible substrate adapted to be coupled to the mouthpiece; and
a plurality of sensors integrated on a flexible printed circuit board (PCB) and coupled to the flexible substrate, the plurality of sensors comprising:
a first photoplethysmography (PPG) sensor coupled along an anterior portion of the flexible substrate, and adapted to be located intraorally along a first anatomical location defined along a mid-line of a maxilla, facing outward to the labial mucosa, directly at a septal branch of a superior labial artery in a front lip, to provide a plurality of first PPG signal, consistently, during sleep of the user relating to a plurality of cardiorespiratory parameters including a heart rate, a respiratory rate, and a blood oxygen saturation of the user, and
a second PPG sensor coupled to a rear portion of the flexible substrate, adapted to be located intraorally along a second anatomical location defined within an oral cavity facing outward toward the labial mucosa, to provide a plurality of second PPG signals, consistently, during sleep of the user relating to a plurality of muscular parameters, including from the Buccinator, the Medial Pterygoid, and the Masseter muscles of the user,
wherein the plurality of muscular parameters based on the plurality of second PPG signals is configured to classify one or more sleep stages of the user, individually, or in combination with the plurality of cardiorespiratory parameters based on the plurality of first PPG signal.

2. The intraoral monitoring device as claimed in claim 1, wherein the first PPG sensor comprises:
at least one first light or infrared (IR) source configured to emit light along the first anatomical location; and
at least one first photodetector operatively coupled to the at least one first light or IR source to measure the light reflected from the first anatomical location to generate the plurality of first PPG signal based on a wavelength of the light reflected to determine the plurality of cardiorespiratory parameters.

3. The intraoral monitoring device as claimed in claim 1, wherein the second PPG sensor comprises:
at least one second light or IR source configured to emit light along the second anatomical location; and
at least one second photodetector operatively coupled to the at least one second light or IR source to measure the light reflected from the second anatomical location to generate the plurality of second PPG signal based on a wavelength of the light reflected to determine the plurality of muscular parameters.

4. The intraoral monitoring device as claimed in claim 1, wherein the plurality of sensors further comprises:
at least one microphone or acoustic sensor coupled along a first side portion of the flexible substrate and adapted to be located intraorally along a third anatomical location within the oral cavity near a soft palate and a uvula, where an airway collapses and sound creates, to measure a snoring intensity of the user.

5. The intraoral monitoring device as claimed in claim 4, wherein the plurality of sensors further comprises:
at least one pH sensor coupled on the flexible substrate proximate to the at least one microphone or acoustic sensor and adapted to be located intraorally along a fourth anatomical location, proximate to the third anatomical location defined within the oral cavity of the user along the maxilla arch closest to the salivary glands/ducts, whereby saliva exists in a substantially higher volume, to measure a pH level of the saliva to monitor sleep quality.

6. The intraoral monitoring device as claimed in claim 1, wherein the plurality of sensors further comprises:
at least one temperature sensor;
at least one pressure sensor; and
at least one accelerometer,
wherein each of the at least one temperature sensor, the at least one pressure sensor, and the at least one accelerometer may be coupled along a second side portion of the flexible substrate.

7. The intraoral monitoring device as claimed in claim 1 further comprising:
a microcontroller integrated with the flexible printed circuit board (PCB) on the flexible substrate to be communicably coupled to the plurality of sensors to analyze data received by the plurality of sensors.

8. The intraoral monitoring device as claimed in claim 7 further comprising:
a Bluetooth module integrated on the flexible printed circuit board (PCB) and coupled to the flexible substrate, proximate to the first PPG sensor, and adapted to be located intraorally along a fifth anatomical location defined along a center of an oral cavity where an amount of saliva is substantially low to transmit the data received by the plurality of sensors or the microcontroller to an external computing device.

9. The intraoral monitoring device as claimed in claim 1 further comprising:
a battery, and
a charging circuitry electrically coupled to the battery,
wherein the battery and the charging circuitry are coupled to the flexible substrate and integrated with the PCB and adapted to be located intraorally along a sixth anatomical location within the oral cavity.

10. A method for obtaining a sleep pattern of a user, the method comprising:
inserting, intraorally, a mouthpiece having an intraoral monitoring device, wherein the intraoral monitoring device comprises a flexible substrate, and a plurality of sensors integrated on a flexible printed circuit board (PCB) and coupled to the flexible substrate, the plurality of sensors comprising: a first photoplethysmography (PPG) sensor coupled along an anterior portion of the flexible substrate, and a second photoplethysmography (PPG) sensor coupled to a rear portion of the flexible substrate;
placing, intraorally, the first PPG sensor along a first anatomical location defined along a mid-line of a maxilla, facing outward toward the labial mucosa, directly at a septal branch of a superior labial artery in a front lip;
obtaining a plurality of first PPG signal from the first PPG sensor, consistently, during sleep of the user relating to a plurality of cardiorespiratory parameters including heart rate, respiratory rate, and blood oxygen saturation of the user;
placing, intraorally, the second PPG sensor along a second anatomical location defined along an interior part of an oral cavity facing outward toward the labial mucosa.
obtaining a plurality of second PPG signals from the second PPG sensor, consistently, during sleep of the user relating to a plurality of muscular parameters, including from the Buccinator, Medial Pterygoid, and Masseter muscles of the user;
classifying one or more sleep stages of the user based on the plurality of muscular parameters based on the plurality of second PPG signals, individually, or in combination with the plurality of cardiorespiratory parameters based on the plurality of first PPG signal, as the plurality of muscular parameters varies according to the one or more sleep stages.

11. The method as claimed in claim 10 further comprising:
measuring a snoring intensity of the user by placing at least one microphone or acoustic sensor coupled along a first side portion of the flexible substrate to be located intraorally along a third anatomical location defined along the interior part of the oral cavity near a soft palate and a uvula, where an airway collapses and sound creates.

12. The method as claimed in claim 11 further comprising:
measuring a pH level of saliva to monitor sleep quality by at least one pH sensor coupled on the flexible substrate proximate to the at least one microphone or acoustic sensor and adapted to be located intraorally along a fourth anatomical location, proximate to the third anatomical location defined within the oral cavity of the user along the maxilla arch closest to the salivary glands/ducts, whereby saliva exists in a substantially higher volume.

13. The method as claimed in claim 10 further comprising:
measuring an intraoral temperature via at least one temperature sensor;
measuring an intraoral pressure via at least one pressure sensor; and
monitoring an intraoral movement via at least one accelerometer,
wherein each of the at least one temperature sensor, the at least one pressure sensor, and the at least one accelerometer are coupled along a second side portion of the flexible substrate.

14. A system for obtaining a sleep pattern of a user, the system comprising:
a network;
one or more computing devices or platforms communicably coupled to the network; and
an intraoral monitoring device for a mouthpiece adapted to be received intraorally in a dentition of a user, the intraoral monitoring device communicably coupled to the one or more computing devices or platforms via the network, the intraoral monitoring device, comprising:
a flexible substrate adapted to be coupled to the mouthpiece; and
a plurality of sensors integrated on a flexible printed circuit board (PCB) and coupled to the flexible substrate, the plurality of sensors comprising:
a first photoplethysmography (PPG) sensor coupled along an anterior portion of the flexible substrate, and adapted to be located intraorally along a first anatomical location defined along a mid-line of a maxilla, facing the labial mucosa, directly at a septal branch of a superior labial artery in a front lip, to provide a plurality of first PPG signal, consistently, during sleep of the user relating to a plurality of cardiorespiratory parameters including a heart rate, a respiratory rate, and a blood oxygen saturation of the user, and
a second photoplethysmography (PPG) sensor coupled to a rear portion of the flexible substrate, adapted to be located intraorally along a second anatomical location defined along an interior part of an oral cavity facing outward toward the labial mucosa, to provide a plurality of second PPG signals, consistently, during sleep of the user relating to a plurality of muscular parameters, including from the Buccinator, Medial Pterygoid, and Masseter muscles of the user,
wherein the plurality of muscular parameters based on the plurality of second PPG signals is configured to classify one or more sleep stages of the user, individually, or in combination with the plurality of cardiorespiratory parameters based on the plurality of first PPG signal, as the plurality of muscular parameters varies according to the one or more sleep stages.

15. The system as claimed in claim 14, wherein the intraoral monitoring device further comprises:
a microcontroller integrated with the flexible printed circuit board (PCB) on the flexible substrate to be communicably coupled to the plurality of sensors to analyze data received by the plurality of sensors.

16. The system as claimed in claim 15, wherein the intraoral monitoring device further comprises:
a Bluetooth module integrated on the flexible printed circuit board (PCB) and coupled to the flexible substrate, proximate to the first PPG sensor, and adapted to be located intraorally along a center of an oral cavity where an amount of saliva is substantially low to transmit the data received by the plurality of sensors or the microcontroller to the one or more computing device or platform via the network.

17. The system as claimed in claim 14, wherein the one or more computing devices or platforms comprise:
one or more computing devices or platforms comprise one or more of a mobile application platform, a device management platform, a data management platform, and a remote patient monitoring platform.
